(19) United States
(12) Patent Application Publication
(10) Pub. No.: US 2025/0064766 A1
(43) Pub. Date: Feb. 27, 2025

(54) COMPOSITIONS AND METHODS FOR INCREASING ARGININE AND NITRIC OXIDE LEVELS

(71) Applicant: Vireo Systems, Inc., Madison, TN (US)

(72) Inventor: Mark C. Faulkner, Madison, TN (US)

(73) Assignee: Vireo Systems, Inc., Madison, TN (US)

(21) Appl. No.: 18/813,684

(22) Filed: Aug. 23, 2024

Related U.S. Application Data
(60) Provisional application No. 63/534,207, filed on Aug. 23, 2023.

(51) Int. Cl. A61K 31/198; A61K 31/706; A61P 43/00

(52) U.S. Cl. CPC A61K 31/198; A61K 31/706; A61P 43/00

(57) ABSTRACT

Non-toxic stimulating agents, supplements and/or pharmaceutical preparations including such stimulating agents for increasing arginine and nitric oxide (NO) levels in the body (above and beyond the naturally occurring levels).

### FIELD OF THE INVENTION

This invention relates to compositions and methods for increasing arginine and nitric oxide (NO) levels in the body (above and beyond the naturally occurring levels). In particular, the present invention is directed to safe, non-toxic stimulating agents, supplements and/or pharmaceutical preparations including such stimulating agents, and methods of increasing arginine and NO levels in the body including via administration of such supplements and/or pharmaceutical preparations. In addition, the supplements and pharmaceutical preparations made in accordance with the present disclosure are contemplated for use in controlling NO metabolism disorders and improving cardiovascular function.

### BACKGROUND OF THE INVENTION

Nitric oxide (NO) plays an important role in cell metabolism, vasodilation, and is also critical in aligning tissue energy demand with supply. In particular, one of the primary functions of NO is to relax/widen the blood vessels, which, in turn, improves blood flow and oxygen supply to tissues and lowers blood pressure. Indeed, at a high level, NO production is essential for overall health because it allows blood, nutrients, and oxygen to travel to every part of the body effectively and efficiently.

NO production occurs naturally in the body. In particular, the body naturally produces NO as a free radical that is the end-product of a conversion process of dietary nitrates and vitamin C. NO deficiency contributes to the development and progression of multiple age- and lifestyle-related diseases. For example, diminished bioavailability of NO may lead to depression, poor vision, fatigue, hypertension, and memory loss. Of course, abnormally high NO levels may also cause issues. For example, high NO levels can, at a minimum, cause headaches, and more concerning, damage on a cellular level, which is associated with neurodegenerative diseases such as Alzheimer disease, Parkinson disease, amyotrophic lateral sclerosis, and Huntington's disease.

While taking certain nitrate-containing drugs, such as glyceryl trinitrate or sodium nitroprusside, or eating certain foods such as beets, poultry, garlic, leafy green vegetables high in nitrates, citrus fruits, nuts and seeds, and cocoa can provide a ready source of NO, NO is an unstable molecule that degrades quickly in the bloodstream, so it must be constantly replenished. As such, there is a need for an improved method of boosting NO production in the body when the diet is poor and/or other factors, such as age, compromise the body's ability to maintain healthy NO levels. One such method involves enriching tissue levels of arginine, an essential precursor molecule required for de novo NO production via NO synthetase (NOS). However, oral L-arginine supplementation has not been effective due to gastrointestinal and hepatic extraction of arginine. And, while oral L-citrulline supplementation has been shown to increase plasma and tissue levels of arginine, such supplementation fails to produce arginine with efficiency (per dose) and results in excess citrulline that must be excreted from the body.

There is a need to identify more effective and efficient forms of supplementation that do not result in excess citrulline that may overload the kidneys and affect creatine production. The present disclosure addresses this need through methods to increase arginine and NO levels in the body and supplements and/or pharmaceutical preparations for use in the methods. Advantageously, without being bound by any particular theory, the methods of the present disclosure may help to lower blood pressure, improve wound healing, improve physical performance, boost reproduction, and improve cognitive ability.

### SUMMARY OF THE INVENTION

Without being bound by any particular theory, the compositions of the present disclosure increase nitric oxide (NO) levels in the body. Relatedly, the compositions of the present disclosure increase plasma concentrations of the major metabolites and precursors of NO including, but not limited to, arginine and citrulline. Moreover, administration of the compositions of the present disclosure results in vasodilation and, as a result, a measurable increase in blood flow and decreased pressure on the blood vessel walls, which, in turn, results in a decrease in blood pressure. Furthermore, administration of the compositions of the present disclosure are contemplated to produce an increase in NO-derived products/metabolites levels in blood and/or urine.

The present disclosure is directed to a composition for increasing nitric oxide physiological levels in a subject, which includes a therapeutically effective amount of a stimulating agent, wherein the stimulating agent includes a pharmaceutically acceptable salt of a direct arginine precursor, an indirect arginine precursor, or a combination thereof. In some embodiments, the direct arginine precursor includes citrulline. In this aspect, the stimulating agent may include citrulline hydrochloride. In other embodiments, the indirect arginine precursor includes glutamine. In this aspect, the stimulating agent may include glutamine hydrochloride. In some aspects, the composition also includes an antioxidant. A nonlimiting example of such an antioxidant is betalain. The composition may include, in some embodiments, a creatine derivative. A nonlimiting example of such a creatine derivative is creatine hydrochloride.

In some embodiments, the composition may be in an oral dosage form. In some aspects, the oral dosage form may be a powder. In other aspects, the oral dosage form is a pill, capsule, tablet, or liquid.

The present disclosure also relates to a composition for increasing arginine physiological levels in a subject, which includes a therapeutically effective amount of a stimulating agent, wherein the stimulating agent includes a pharmaceutically acceptable salt of a direct arginine precursor, an indirect arginine precursor, or a combination thereof, and wherein the stimulating agent has an aqueous solubility of at least two times that of L-citrulline. In some aspects, the stimulating agent has a solubility of about 120 mg/ml to about 200 mg/ml in water at 62° F. In some aspects, the direct arginine precursor includes citrulline. In some embodiments, the stimulating agent includes citrulline hydrochloride. In other embodiments, the composition also includes an antioxidant. The antioxidant may include, but is not limited to, betalain. In still other embodiments, the composition may include a creatine derivative, e.g., creatine hydrochloride. In other aspects, the composition may be included in an oral dosage form such as a powder, pill, capsule, tablet, or liquid.

The present disclosure also relates to a method of increasing the nitric oxide physiological levels in an animal including:
providing a composition including a stimulating agent, wherein the stimulating agent includes a pharmaceutically acceptable salt of a direct arginine precursor, an indirect arginine precursor, or a combination thereof, and wherein the stimulating agent has an aqueous solubility of at least two times that of L-citrulline; and administering an effective dose of the composition to the animal. In some embodiments, the direct arginine precursor includes citrulline. In this aspect, the stimulating agent may include citrulline hydrochloride. In other embodiments, the indirect arginine precursor includes glutamine. In this aspect, the stimulating agent may include glutamine hydrochloride. In some embodiments, the composition may be included in an oral dosage form. In some aspects, the oral dosage form is a powder. In other aspects, the oral dosage form is a pill, capsule, tablet, or liquid. In still other embodiments, the composition further includes at least one additive including one or more antioxidants, creatine derivatives, or combinations thereof.

The present disclosure also relates to a method of increasing arginine physiological levels in an animal including:
providing a composition including a stimulating agent, wherein the stimulating agent includes a pharmaceutically acceptable salt of a direct arginine precursor, an indirect arginine precursor, or a combination thereof, and wherein the stimulating agent has an aqueous solubility of at least two times that of L-citrulline; and
administering an effective dose of the composition to the animal, wherein the composition has an arginine conversion efficiency according to (I(b)):

$${ACE}_{plasma} = \frac{{Arg}_{pl}}{{Cit}_{pl}} \geq 0 \text{.30} \qquad ( I ( b ) )$$

wherein Argₚₗ represents the dose normalized level of arginine in plasma and Citₚₗ represents the dose normalized level of citrulline in plasma. In some aspects, the direct arginine precursor includes citrulline. In other aspects, the stimulating agent includes citrulline hydrochloride. In some embodiments, the composition may be included in an oral dosage form. In some aspects, the oral dosage form is a powder. In other aspects, the oral dosage form is a pill, capsule, tablet, or liquid. In still other embodiments, the composition further includes at least one additive including one or more antioxidants, creatine derivatives, or combinations thereof. In yet other embodiments,

$${ACE}_{plasma} = \frac{{Arg}_{pl}}{{Cit}_{pl}} \geq 1.0 .$$

The present disclosure also relates to a method of increasing nitric oxide physiological levels in an animal including:
providing a composition including an effective dose of stimulating agent, wherein the stimulating agent includes a pharmaceutically acceptable salt of a direct arginine precursor and has an aqueous solubility of about 120 mg/ml to about 200 mg/ml in water at 62° F.; and
administering the composition to the animal, wherein the composition has an arginine conversion efficiency according to (I(b)):

$${ACE}_{plasma} = \frac{{Arg}_{pl}}{{Cit}_{pl}} \geq 0 \text{.15} \qquad ( I ( b ) )$$

wherein Argₚₗ represents the dose normalized level of arginine in plasma and Citₚₗ represents the dose normalized level of citrulline in plasma. In some embodiments, the composition is included in an oral dosage form. In other embodiments, the effective dose ranges from about 1000 mg to about 3000 mg. In still other embodiments, the stimulating agent includes citrulline hydrochloride. In yet other embodiments, the composition further includes creatine hydrochloride, and wherein the composition has an aqueous solubility of about 500 mg/ml to about 1000 mg/ml in water at 62° F.

### BRIEF DESCRIPTION OF THE DRAWINGS

Further features and advantages of the invention can be ascertained from the following detailed description that is provided in connection with the drawings described below:

FIG. 1 is a graph showing the solubility of compositions made in accordance with the present disclosure;

FIG. 2 is a graphical illustration of the arginine levels in urine after administration of the compositions of the present disclosure;

FIG. 3 is a graphical illustration of citrulline levels in urine after administration of the compositions of the present disclosure;

FIG. 4 is a graphical illustration of the ratio of arginine to citrulline in urine after administration of the compositions of the present disclosure;

FIGS. 5-6 are graphical illustrations of the arginine levels in plasma after administration of the compositions of the present disclosure;

FIG. 7 is a graphical illustration of citrulline levels in plasma after administration of the compositions of the present disclosure; and

FIG. 8 is a graphical illustration of the ratio of arginine to citrulline in plasma after administration of the compositions of the present disclosure.

### DETAILED DESCRIPTION OF THE INVENTION

The present disclosure is directed to methods for increasing nitric oxide (NO) levels in the body and the stimulating agents and compositions useful in the method. In particular, the present disclosure provides a method for increasing NO levels in the body by way of increased production of arginine levels in the body. The produced arginine is then available for uptake within the vasculature endothelial cells where it can be converted to NO via various forms of NOS. The locally produced NO is then available to act locally to dilate the blood vessels and allow increased blood flow to the tissues, which, in turn, increases oxygen delivery to cells.

The stimulating agent(s) that increases the arginine levels and compositions including such stimulating agents are also discussed. In one embodiment, the method of the present disclosure includes administering a supplement or pharmaceutical preparation that includes the stimulating agents of the present disclosure to an animal in order to increase glutamine, citrulline, arginine, and/or NO levels in the body.

Without being bound by any particular theory, the administration of a composition including at least one of the stimulating agents discussed herein may be used as an arginine precursor synthesized de novo in the kidneys and to ultimately boost nitrogen production in the body. More specifically, oral administration of compositions including at least one stimulating agent as discussed herein are useful in enhancing systemic citrulline and arginine production and nitrogen balance.

### Stimulating Agents

Without being bound by any particular theory, the stimulating agent(s) disclosed herein is believed to play an important role in the metabolism and regulation of NO. Thus, in some aspects, a supplement or pharmaceutical composition including the stimulating agent(s) of the present disclosure may be used to address NO metabolism disorders and/or improve cardiovascular function. In addition, the use of supplements or pharmaceutical compositions including at least one stimulating agent and, optionally, an additive, made in accordance with the present disclosure may stimulate muscle protein synthesis in the elderly population. It is believed that oral supplementation with supplements or pharmaceutical compositions made in accordance with the present disclosure are more effective in increasing nitric oxide levels than oral supplementation with arginine itself. As such, the use of the compositions discussed herein may be particularly useful in circumstances or conditions in which elevating circulating arginine levels has a beneficial effect and/or where arginine supplementation may be considered harmful or contraindicated.

Suitable stimulating agents of the present disclosure include the pharmaceutically acceptable salts of direct and/or indirect arginine precursors. In this aspect, pharmaceutically acceptable salts include, for example, alkali metal salts and addition salts of free acids or free bases. Suitable pharmaceutically-acceptable acid addition salts may be prepared from an inorganic acid or from an organic acid. Non-limiting examples of suitable inorganic acids include hydrochloric acid, hydrobromic acid, hydroiodic acid, nitric acid, carbonic acid, sulfuric acid, phosphoric acid, and combinations thereof. Suitable organic acids include, but are not limited to, aliphatic, cycloaliphatic, aromatic, heterocyclic, carboxylic and sulfonic classes of organic acids, such as, for example, formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, stearic, algenic, β-hydroxybutyric, cyclohexylaminosulfonic, galactaric, and galacturonic acid, and combinations thereof. Non-limiting examples of suitable pharmaceutically-acceptable base addition salts include metallic salts made from aluminum, calcium, lithium, magnesium, potassium, sodium and zinc or organic salts made from primary, secondary and tertiary amines, cyclic amines, N,N′-dibenzylethylenediamine, chloroprocaine, choline, diethanolamine, ethylenediamine, meglumine (N-methylglucamine), procaine, and combinations thereof.

In some embodiments, the salt is hydrochloride (HCl), hydrobromide (HBr), acetate, benzoate, besylate, bitartrate, carbonate, bromide, citrate, estolate, gluconate, lactate, malate, mesylate, stearate, tartrate, valerate, nitrate, pamoate, or a combination thereof. In one particular embodiment, the salt is hydrochloride.

The stimulating agent may have a solubility in water at 62° F. of about 120 mg/ml to about 200 mg/ml. In other embodiments, the salt of a direct arginine precursor has a solubility in water at 62° F. of about 130 mg/ml to about 190 mg/ml. In still other embodiments, the salt of a direct arginine precursor has a solubility in water at 62° F. of about 130 mg/ml to about 150 mg/ml. In yet other embodiments, the salt of a direct arginine precursor has a solubility in water at 62° F. of about 145 mg/ml to about 160 mg/ml.

Supplementation with a stimulating agent that is a salt of a direct arginine precursor promotes the metabolism of citrulline to arginine in the kidneys, which, in turn, boosts NO production in the body. In some aspects, the direct arginine precursor is citrulline. In this aspect, the stimulating agent may be a citrulline salt. In some embodiments, the citrulline salt has a solubility in water at 62° F. of about 1.5 to 5 times that of L-citrulline. In one aspect, the citrulline salt has a solubility in water at 62° F. that is about 2 to about 3.5 times that of L-citrulline. In another aspect, the citrulline salt has a solubility in water at 62° F. that is about 2.2 to about 3.4 times that of L-citrulline. For example, the citrulline salt may be citrulline hydrochloride, which has a solubility in water at 62° F. that is 3 times that of L-citrulline. In other embodiments, the stimulating agent is at least one of citrulline hydrobromide, citrulline acetate, citrulline benzoate, citrulline besylate, citrulline bitartrate, citrulline carbonate, citrulline bromide, citrulline citrate, citrulline estolate, citrulline gluconate, citrulline lactate, citrulline malate, citrulline mesylate, citrulline stearate, citrulline tartrate, citrulline valerate, citrulline nitrate, or citrulline pamoate.

In some embodiments, the salt of a direct arginine precursor has a solubility in water at 62° F. of about 130 mg/ml to about 200 mg/ml. In other embodiments, the salt of a direct arginine precursor has a solubility in water at 62° F. of about 140 mg/ml to about 190 mg/ml. In still other embodiments, the salt of a direct arginine precursor has a solubility in water at 62° F. of about 145 mg/ml to about 175 mg/ml. In yet other embodiments, the salt of a direct arginine precursor has a solubility in water at 62° F. of about 145 mg/ml to about 160 mg/ml.

Supplementation with a stimulating agent that is a salt of an indirect arginine precursor will promote the synthesis of citrulline in the intestine, which, in turn, will promote the citrulline metabolism to arginine in the kidneys, which, in turn, will boost NO production in the body. In this aspect, the indirect arginine precursor may be glutamine. In some aspects, the indirect arginine precursor is a glutamine salt. In certain embodiments, the glutamine salt is glutamine hydrochloride. In other embodiments, the stimulating agent is at least one of glutamine hydrobromide, glutamine acetate, glutamine benzoate, glutamine besylate, glutamine bitartrate, glutamine carbonate, glutamine bromide, glutamine citrate, glutamine estolate, glutamine gluconate, glutamine lactate, glutamine malate, glutamine mesylate, glutamine stearate, glutamine tartrate, glutamine valerate, glutamine nitrate, or glutamine pamoate.

In some embodiments, the salt of an indirect arginine precursor has a solubility in water at 62° F. of about 120 mg/ml to about 180 mg/ml. In other embodiments, the salt of an indirect arginine precursor has a solubility in water at 62° F. of about 130 mg/ml to about 170 mg/ml. In still other embodiments, the salt of an indirect arginine precursor has a solubility in water at 62° F. of about 135 mg/ml to about 160 mg/ml. In yet other embodiments, the salt of an indirect arginine precursor has a solubility in water at 62° F. of about 135 mg/ml to about 150 mg/ml.

In other embodiments, at least two stimulating agents are used. In this aspect, a first stimulating agent may be a salt of a direct arginine precursor and a second stimulating agent may be at least one salt of an indirect arginine precursor. For example, a composition may include a citrulline salt and a glutamine salt. In this regard, the composition may have a solubility in water at 62° F. of about 130 mg/ml to about 200 mg/ml. In other embodiments, the salt of a direct arginine precursor has a solubility in water at 62° F. of about 140 mg/ml to about 190 mg/ml. In still other embodiments, the salt of a direct arginine precursor has a solubility in water at 62° F. of about 145 mg/ml to about 175 mg/ml. In yet other embodiments, the salt of a direct arginine precursor has a solubility in water at 62° F. of about 145 mg/ml to about 160 mg/ml.

As a result of the higher solubility (as compared to the parent compound), certain stimulating agents can be dosed in a dietary supplement or pharmaceutical preparation in a lower amount. For example, if L-citrulline is dosed at about 2 grams per 100 pounds of body weight, an effective dose of certain citrulline salts may be dosed at about 1 g per 100 pounds of body weight or less.

In addition, the stimulating agents of the present disclosure have good oral absorption properties. As used herein, the term “bioavailability” refers to the rate and amount of a substance that reaches the systemic circulation of a subject following administration of the substance or form of the substance to the subject. By definition, when a composition is administered intravenously, its bioavailability is 100 percent. However, when a composition is administered via other routes (such as orally), its bioavailability decreases (due to incomplete absorption and first-pass metabolism). More specifically, bioavailability is a measure of the ratio of the amount of substance “absorbed” from a test formulation to the amount “absorbed” after administration of a standard formulation. Frequently, the “standard formulation” used in assessing bioavailability is the aqueous solution of the substance, given intravenously.

Accordingly, bioavailability is one of the principal pharmacokinetic properties of substances and can be determined by evaluating, for example, the plasma or blood concentration-versus-time profile for a substance. Parameters useful in characterizing a plasma or blood concentration-versus-time curve include the area under the curve (AUC), the maximum concentration (Cₘₐₓ), and the time to maximum concentration (Tₘₐₓ). As used herein, the term “AUC” refers to the area under a curve representing the concentration of a compound or metabolite thereof in a biological fluid, e.g., plasma and blood, in a patient or subject as a function of time following administration of the compound to the subject. The AUC may be determined by measuring the concentration of a compound or metabolite thereof in a biological fluid using methods such as liquid chromatography-tandem mass spectrometry (LC/MS/MS), at various time intervals, and calculating the area under the plasma concentration-versus-time curve. Suitable methods for calculating the AUC from a concentration-versus-time curve are well known in the art. Cₘₐₓ is the maximum concentration of a drug in the plasma or blood of a patient or subject following administration of a dose of the substance or form of the substance to the patient or subject. Tₘₐₓ is the time to the maximum concentration (Cₘₐₓ) of a substance in the plasma or blood of a patient or subject following administration of a dose of the substance or form of the substance to the patient or subject.

The amount of substance absorbed is taken as a measure of the ability of the formulation to deliver the substance to the sites of action. Obviously-depending on such factors as disintegration and dissolution properties of the dosage form, and the rate of biotransformation relative to the rate of absorption-dosage forms containing identical amounts of active substance may differ markedly in their abilities to make the substance available, and therefore, in their abilities to permit the substance to manifest its expected pharmacodynamic and therapeutic properties. The “amount absorbed” is conventionally measured by one of two criteria, either the area under the plasma concentration (AUC) or the total (cumulative) amount of the substance excreted in the urine following drug administration. A linear relationship exists between the AUC and dose when the fraction of the drug absorbed is independent of dose, and elimination rate (half-life) and volume of distribution are independent of dose and dosage form. However, when AUC is dependent on dose, as occurs when, for example, there is saturable absorption, significant metabolism, or poor solubility of the substance in the GI tract, a non-linear relationship exists between AUC and dose.

In order to assess the relative bioavailability of the stimulating agents (and to correct for the slightly different doses administered with various forms due to the different molecular weights), the AUC plasma uptake values observed for the standard (here, creatine monohydrate) and each formulation are entered into the following equation to produce a ratio:

$$\frac{{AUC}_{SampleA} \times {Dose}_{B}}{{AUC}_{SampleB} \times {Dose}_{A}}$$

Based on this relationship, the relative bioavailability of any of the stimulating agents to the parent compound (non-salt form) is about 1.3 or greater. In some embodiments, the relative bioavailability of any of the formulations to the parent compound is about 1.5 or greater. In other embodiments, the relative bioavailability of any of the stimulating agents to the parent compound is about 1.6 or greater. In still other embodiments, the relative bioavailability of any of the stimulating agents to the parent compound is about 1.7 or greater.

In other words, the relative bioavailability of the stimulating agents is at least about 30 percent greater than the parent compound (non-salt form), about 40 percent greater than the parent compound, or about 50 percent greater than the parent compound. In one embodiment, the bioavailability of the stimulating agents is at least about 50 percent greater than bioavailability of the parent compound. In another embodiment, the bioavailability of the stimulating agents is at least about 65 percent greater than bioavailability of the parent compound. In yet another embodiment, the stimulating agents have a bioavailability of at least about 70 percent greater relative to the parent compound.

### Additives

The compositions of the present disclosure may also include various additives. The additives may include phytochemicals, creatine derivatives, and combinations thereof. The additive(s) may be present in the composition in an amount of about 10 percent to about 85 percent by weight based on the total weight of the composition. In some embodiments, the additive(s) may be present in the composition in an amount of about 25 percent to about 85 percent by weight based on the total weight of the composition. In other embodiments, the composition may include about 35 percent to about 85 percent by weight of additives (based on the total weight of the composition). In still other embodiments, the additives are present in an amount of about 45 percent to about 75 percent by weight based on the total weight of the composition.

For example, the composition may include one or more phytochemicals. Non-limiting examples of suitable phytochemicals include antioxidants, flavonoids, flavones, isoflavones, catechins, anthocyanidins, isothiocyanates, carotenoids, allyl sulfides, monophenols, polyphenols, terpenes, limonoids, lipids, phytosterols, betalains, organosulfides, indoles, glucosinolates, beta carotene, ascorbic acid (vitamin C), folic acid, and vitamin E.

In some embodiments, the composition includes at least one antioxidant. Without being bound by any particular theory, the presence of an antioxidant in the composition may help to increase the stability of the NO that is produced as a result of the administration of the compositions of the present disclosure and, thus, limit the breakdown of the NO molecules. More specifically, when antioxidants are included in the compositions of the present disclosure, the free radicals that contribute to the short life of the NO molecules are neutralized. In some embodiments, the antioxidant(s) is included in the composition in an amount of about 500 mg to about 2500 mg. In other embodiments, the composition includes about 750 mg to about 2000 mg of at least one antioxidant. Suitable antioxidants include, but are not limited to, betalain, conjugated linoleic acid, iron, L-arginine, pycnogenol, quercetin, alpha-lipoic acid, and combinations thereof. For example, the composition may include beet root powder, which contains betalain.

In another aspect, the composition includes phytosterol, polyphenol, or combinations thereof. Non-limiting examples of sources of such phytochemicals include curcumin, coumarins, rosemarinic acid, cacao powder, saffron, oregano, rosemary, and cloves. In some embodiments, the composition includes polyphenols such as flavonoids, phenolic acids, lignans, stilbenes, and combinations thereof.

In still another aspect, the composition includes one or more creatine derivatives. Suitable creatine derivatives include, but are not limited to creatine hydrochloride, creatine ethyl ester, creatine glycerol laurate ester, creatine propylene glycol laurate ester, creatine amide tert butyl ester, dicreatine glycerol ester, tricreatine glycerol ester, betaine cyclopentyl ester, creatine mesylate, creatine citrate, creatine pyruvate, or combinations thereof. The creatine derivative may present in the composition in an amount of about 250 mg to about 2400 mg. In another embodiment, the composition may include about 750 mg to about 1500 mg of the creatine derivative.

In another aspect, the stimulating agent(s) and one or more creatine derivative may be present in the composition in a ratio of 0.5:1.50 to 1.50:0.50. In one embodiment, the ratio of stimulating agent(s) to the one or more creatine derivatives in the composition ranges from 0.75:1.25 to 1.25:0.75. In another embodiment, the ratio of stimulating agent(s) to the one or more creatine derivatives in the composition ranges from 0.9:1.1 to 1.1:0.9. For example, the ratio of stimulating agent(s) to the one or more creatine derivatives in the composition may be 1:1.

Without being bound by any particular theory, the solubility of a composition with one or more stimulating agents and one or more creatine derivatives to a composition is higher than the solubility of a composition that includes only one or more stimulating agents. In some embodiments, the solubility of a composition with one or more stimulating agents and one or more creatine derivatives to a composition is at least about 2 times higher than the solubility of a composition that includes only one or more stimulating agents. In other embodiments, the solubility of a composition with one or more stimulating agents and one or more creatine derivatives to a composition is at least about 3 times higher than the solubility of a composition that includes only one or more stimulating agents. In yet other embodiments, the solubility of a composition with one or more stimulating agents and one or more creatine derivatives to a composition is at least about 4 times higher than the solubility of a composition that includes only one or more stimulating agents.

In some embodiments, the solubility in water at 62° F. of is about 500 mg/ml to about 1000 mg/ml. In other embodiments, a composition with one or more stimulating agents and one or more creatine derivatives has a solubility of in water at 62° F. of about 550 mg/ml to about 980 mg/ml. In still other embodiments, the solubility in water at 62° F. of a composition with one or more stimulating agents and one or more creatine derivatives is about 575 mg/ml to about 950 mg/ml. In yet other embodiments, the solubility in water at 62° F. of a composition with one or more stimulating agents and one or more creatine derivatives to a composition is about 590 mg/ml to about 750 mg/ml.

Similarly, the relative bioavailability of a composition with one or more stimulating agents and one or more creatine derivatives to a composition that includes only one or more stimulating agents may be about 1.5 or greater. In some embodiments, the relative bioavailability of any of the formulations to the parent compound is about 1.7 or greater. In other embodiments, the relative bioavailability of any of the stimulating agents to the parent compound is about 2.0 or greater. In still other embodiments, the relative bioavailability of any of the stimulating agents to the parent compound is about 2.5 or greater.

Other additives include, but are not limited to, proteins, peptides, simple and complex carbohydrates, lipids, fats, fibers, or combinations thereof may be included in the compositions discussed herein. Suitable proteins include serum albumin such as human serum albumin (HSA), recombinant human albumin (rHA), gelatin, casein, and combinations thereof. Suitable carbohydrates include, but are not limited to monosaccharides such as fructose, maltose, galactose, glucose, D-mannose, D-ribose, sorbose, and combinations thereof; disaccharides, such as lactose, sucrose, trehalose, cellobiose, and combinations thereof; polysaccharides, such as raffinose, melezitose, maltodextrins, dextrans, starches, and combinations thereof; and alditols, such as mannitol, xylitol, maltitol, lactitol, xylitol, sorbitol (glucitol), myoinositol, and combinations thereof.

The composition may also include a homeopathic compound, a co-medication, a nutraceutical, a plant extract, an herbal preparation, a cosmetic agent, a pharmaceutical, or combinations thereof. Suitable homeopathic compounds include, but are not limited to, actaca Spicata, Aesculus hippocastanum, Arnica montana, Belladonna, Bellis perennis, Bryonia, calcarea carbonica, calcarea fluorica, calc sulph MM, causticum, cayenne, Cimicifuga racemosa, formicum acidum, Hamamelis virginiana, Hypericum perforatum, magnesia phosphorica, Phytolacca decandra, Pulsatilla, Rhododendron chrysanthum, Rhus toxicodendron, Ruta graveolens, salicylicum acidum, Sepia, sulphu, turmeric, green tea extract, grape extract, Foeniculum vulgare, Bellis perrinis, Boswellia serrate, bromeliacaea, devil's claw (Harpagophytum procumbens), bromelain, cordyalis yanhusuo, or combinations thereof. In addition, the composition may include vitamins such as vitamins C, D, and E.

The composition may further include at least one of any suitable auxiliaries including, but not limited to, diluents, binders, stabilizers, buffers, salts, lipophilic solvents, preservatives, adjuvants, or the like. For example, in some embodiments, the composition may include a surfactant, alone or in combination with other surfactants, to stabilize the formulation. The surfactant can be selected from a variety of known anionic surfactants, cationic surfactants, zwitterionic surfactants, nonionic surfactants, surface active biological modifiers, and combinations thereof. Suitable buffers include, but are not limited to, organic acid salts such as salts of citric acid, ascorbic acid, gluconic acid, carbonic acid, tartaric acid, succinic acid, acetic acid, or phthalic acid; Tris, tromethamine hydrochloride, or phosphate buffers.

The composition may also contain pharmaceutically acceptable carriers such as coloring agents, emulsifying agents, suspending agents, ethanol, EDTA or similar chelating agents, citrate buffer, water, and combinations thereof. Moreover, the compositions may include polymeric excipients/additives such as polyvinylpyrrolidones, ficolls, dextrates, polyethylene glycols, flavoring agents, anti-microbial agents, antioxidants, anti-static agents, steroids, and chelating agents.

### Administration

In one embodiment, the composition is administered to a subject. A subject may include, but is not limited to, a human, a canine, and an animal. Dosages range from use “as needed” to daily dosages of 1-2 doses taken 1-4 times daily. In this aspect, a composition including an effective amount of the stimulating agent may be administered once or twice daily. In another embodiment, the composition containing an effective amount of the stimulating agent may be administered multiple doses in a day, e.g., two to four doses. For example, in some embodiments, the composition containing an effective amount of the stimulating agent may be administered in three separate doses per day. In another embodiment, the composition containing an effective amount of the stimulating agent may be administered in four separate doses per day.

The daily effective amount of stimulating agent is as needed/desired for blood flow health. As such, the daily effective amount needed to achieve the desired blood flow health may vary based on preexisting conditions, age, performance applications, and combinations thereof. For example, if the daily effective amount of stimulating agent is from about 250 mg to about 2400 mg per 100 pounds of body weight, a subject up to 250 pounds may receive one dose of the composition containing about 6250 mg to about 6000 mg of the stimulating agent daily or receive two separate doses of the composition containing about 315 mg to about 3000 mg of the stimulating agent. In another embodiment, if the daily effective amount of stimulating agent is from about 500 to about 1500 mg per 100 pounds of body weight, a subject up to 250 pounds may receive three separate doses of the composition, each containing about 500 mg to about 1000 mg of stimulating agent, per day. In yet another embodiment, if the daily effective amount of stimulating agent is from about 500 to about 1500 mg per 100 pounds of body weight, a subject up to 250 pounds may receive four doses of the composition, each containing about 375 mg to 750 mg of stimulating agent, per day.

The methods of administration of the composition described herein to the subject may vary. In some embodiments, the composition is administered to the subject orally. For example, the composition may be encapsulated or tableted for a solid oral dosage form. In one embodiment, the composition may be administered in the form of a pill, tablet, capsule, or gel capsule.

Solid oral dosage forms for oral administration can include capsules, sustained-release capsules, tablets, sustained release tablets, chewable tablets, sublingual tablets, effervescent tablets, pills, powders, liquids, granules and gels. In such solid dosage forms, the composition can include at least one inert diluent such as sucrose, lactose or starch. Such dosage forms can also include lubricating agents such as magnesium stearate and, in the case of capsules, tablets, effervescent tablets, and pills, the dosage forms can also include buffering agents. Soft gelatin capsules can be prepared to contain a mixture of the composition and vegetable oil. Hard gelatin capsules can contain granules of the composition in combination with a solid, pulverulent carrier such as lactose, saccharose, sorbitol, mannitol, potato starch, corn starch, amylopectin, cellulose derivatives of gelatin.

In some embodiments, the composition is provided orally to the subject in a liquid or powder form. For example, the composition may be in the form of a powder suitable for mixing with water or other liquids. In this aspect, the composition may be added into a beverage or may be provided as an ingredient premixed in a beverage, i.e., a ready-to-drink beverage. For example, if the composition is in the form of a ready-to-drink beverage, it is contemplated that the beverage will have a pH that ranges from 2.5 to 4. In addition, the beverage is stable at room temperature for up to 180 days, i.e., at least about 75 percent of the stimulating agent originally present in the composition (at Day 1) is still present at Day 180 or for longer.

The composition may also be administered as an elixir or as a solution composition. In still another embodiment, the composition may be administered in the form of a functional food, for example, a shake or bar.

In one embodiment, an effective oral dosage of the composition ranges from about 250 mg to 2400 mg per day, or about 3 mg/kg to about 30 mg/kg. In another embodiment, an effective oral dosage ranges from about 400 mg to about 800 mg, or about 5 mg/kg to about 10 mg/kg. In yet another embodiment, an effective oral dosage ranges from about 400 mg to about 1200 mg, or about 5 mg/kg to about 15 mg/kg.

In some embodiments, the effective oral dosage of the composition includes about 500 mg to about 2400 mg of the stimulating agent. In other embodiments, the effective oral dosage includes about 600 mg to about 2000 mg of the stimulating agent. In still other embodiments, the effective oral dosage includes about 1000 mg to about 1500 mg of the stimulating agent. In yet other embodiments, the effective oral dosage of the composition includes about 750 mg to about 1425 mg of the stimulating agent and about 750 mg to about 3000 mg of at least one additive.

In other aspects, the effective oral dosage of the composition includes about 1000 mg to about 7000 mg of the stimulating agent. In other embodiments, the effective oral dosage includes about 1500 mg to about 6500 mg of the stimulating agent. In still other embodiments, the effective oral dosage includes about 2000 mg to about 6000 mg of the stimulating agent. In yet other embodiments, the effective oral dosage includes about 1000 mg to about 3000 mg of the stimulating agent.

In another embodiment, the composition is administered to the subject intravenously. In fact, as briefly discussed above, intravenous administration translates to 100 percent bioavailability and may be particularly suitable when the composition is co-administered with other therapies that are already administered intravenously. In one embodiment, the composition is administered to a subject with a need for adjusted plasma arginine levels intravenously for up to 5 days. In another embodiment, the composition is administered to a subject intravenously for up to 10 days. In another embodiment, the composition is administered to a subject intravenously for up to 15 days.

### Increased Nitric Oxide Production

As discussed previously, NO is involved in many physiological processes including regulation of blood pressure, as well as immune response and neural communication. However, NO has an extremely short physiological half-life. As such, accurate detection and quantification of NO production in the body (particularly before and administration of the compositions of the present disclosure) may be through the quantification of certain NO-derived products and/or metabolites including, but not limited to, nitrite (NO₂⁻), nitrate (NO₃⁻), S-nitrosothiols (RSNOs), N-nitrosamines (RNNOs), and nitrosyl-heme.

In one embodiment, the method of the present disclosure includes administering a supplement or pharmaceutical preparation that includes the stimulating agents of the present disclosure to an animal in order to increase glutamine, citrulline, arginine, and/or NO levels in the body. In this aspect, administration of the compositions of the present disclosure are contemplated to produce an increase in nitrate levels in blood and/or urine. Similarly, administration of the compositions of the present disclosure are contemplated to produce an increase in nitrite levels in urine. Likewise, there may be an increase of RSNOs, RNNOs, and/or nitrosyl-heme as a result of administration of the compositions of the present disclosure.

In some embodiments, administration of the compositions of the present disclosure are also contemplated to produce an efficient increase of arginine production. In this aspect, arginine production may be demonstrated by the arginine urine levels 24 hours after administration and normalizing for the citrulline dose. For example, administration of an effective oral dose of a composition of the present disclosure may result in arginine urine levels per citrulline dose that are higher than conventional L-citrulline. In some aspects, the arginine urine levels 24 hours of administration of an effective oral dose of a composition of the present disclosure are higher than the arginine levels 24 hours after administration with L-citrulline by a factor of about 1.25 to about 3. In other aspects, the arginine urine levels 24 hours of administration of an effective oral dose of a composition of the present disclosure are higher than arginine urine levels after dosing with L-citrulline by a factor of about 1.5 to about 2.5. Moreover, while citrulline urine levels are also expected to increase after dosing with compositions of the present disclosure, the citrulline urine levels 24 hours after dosing (from baseline) are preferably comparable to or less than after dosing with L-citrulline. In some aspects, the citrulline urine levels after administration of an effective oral dose of a composition of the present disclosure are about 80 percent or less of the citrulline urine levels after administration of L-citrulline. In other aspects, the citrulline urine levels after administration of an effective oral dose of a composition of the present disclosure are about 70 percent or less of the citrulline urine levels after administration of L-citrulline. In still other aspects, the citrulline urine levels after administration of an effective oral dose of a composition of the present disclosure are about 60 percent or less of the citrulline urine levels after administration of L-citrulline. In this aspect, the effective oral dose ranges from about 2 g to about 6 g.

Administration of the compositions of the present disclosure may result in arginine urine levels of about 1.5 or more (dose normalized (mmol/mmol)). As would be understood by a person of ordinary skill in the art, the terms “dose normalized” or “normalized per dose” generally refer to the calculation performed with pharmacokinetic parameters, e.g., where the PK parameters are divided by the administered dose. In one embodiment, the compositions of the present disclosure may result in arginine urine levels of about 2.0 or more (dose normalized (mmol/mmol)). In another embodiment, the compositions of the present disclosure may result in arginine urine levels of about 1.5 to about 5.0 (dose normalized (mmol/mmol)). In still another embodiment, the compositions of the present disclosure may result in arginine urine levels of about 1.5 to about 4.0 (dose normalized (mmol/mmol)).

In other embodiments, administration of the compositions of the present disclosure are also contemplated to produce an increase of arginine and/or citrulline levels in plasma. For example, administration of an effective oral dose of a composition of the present disclosure may result in arginine levels in plasma that are higher than the arginine levels in plasma after dosing with L-citrulline. In this aspect, the arginine levels in plasma after dosing with a composition of the present disclosure are higher than the arginine levels in plasma after dosing with L-citrulline by a factor of at least about 1.1. In another aspect, the arginine levels in plasma after dosing with a composition of the present disclosure are higher than the arginine levels in plasma after dosing with L-citrulline by a factor of at least about 1.5. In still another aspect, the peak arginine levels in plasma after dosing with a composition of the present disclosure are higher than the peak arginine levels in plasma after dosing with L-citrulline by a factor of at least about 2.0. In yet other aspects, the arginine levels in plasma after dosing with a composition of the present disclosure are higher than the arginine levels in plasma after dosing with L-citrulline by a factor of at least about 2.2.

Administration of the compositions of the present disclosure may result in arginine plasma levels of about 550 or more (dose normalized (mmol/mmol)). In one embodiment, the compositions of the present disclosure may result in arginine plasma levels of about 600 or more (dose normalized (mmol/mmol)). In another embodiment, the compositions of the present disclosure may result in arginine plasma levels of about 550 to about 1300 (dose normalized (mmol/mmol)). In still another embodiment, the compositions of the present disclosure may result in arginine plasma levels of about 600 to about 1200 (dose normalized (mmol/mmol)).

The compositions of the present disclosure have an overall arginine conversion efficiency (ACE) that is improved over conventional forms of supplementation (e.g., L-citrulline) and can be demonstrated by the following relationship:

$$ACE = {ArgCE}_{urine} + {rg ACE}_{plasma} \qquad ( I )$$

where ACEᵤᵣᵢₙₑ is the arginine conversion efficiency as measured from the arginine (Argᵤᵣ) and citrulline (Citᵤᵣ) in the urine 24 hours after dosing with an effective oral dose (dose normalized):

$${ACE}_{urine} = \frac{{Arg}_{ur}}{{Cit}_{ur}} \qquad ( I ( a ) )$$

and ACEₚₗₐₛₘₐ is the arginine conversion efficiency as measured from the plasma levels of arginine (Argₚₗ) and citrulline (Citₚₗ) after dosing with an effective oral dose (dose normalized):

$${ACE}_{plasma} = \frac{{Arg}_{pl}}{{Cit}_{pl}} \qquad ( I ( a ) )$$

Without being bound by any particular theory, the compositions of the present disclosure produce more arginine with less citrulline as an input or output as compared to conventional supplements intended to increase citrulline or arginine levels in the body. In other words, supplementation with the compositions of the present disclosure result in higher arginine production and lower amounts of citrulline in the plasma (and, at least with lower effective doses of the composition of the present disclosure, in the urine).

In some embodiments,

ACEᵤᵣᵢₙₑ≥0.7

In other embodiments,

ACEᵤᵣᵢₙₑ≥0.9

In still other embodiments,

ACEᵤᵣᵢₙₑ≥1.0

In yet other embodiments,

ACEᵤᵣᵢₙₑ≥1.4

In other embodiments,

ACEᵤᵣᵢₙₑ≥1.8

In still other embodiments,

ACEᵤᵣᵢₙₑ≥2.0

In other embodiments,

ACEₚₗₐₛₘₐ≥0.15

In yet other embodiments,

ACEₚₗₐₛₘₐ≥0.30

In other embodiments,

ACEₚₗₐₛₘₐ≥1.0

In still other embodiments,

ACEₚₗₐₛₘₐ≥1.5

In still other embodiments,

ACEₚₗₐₛₘₐ≥2.0

The overall arginine conversion efficiency (ACE) may be about 0.9 or more. In some embodiments, ACE is about 1.0 or more. In other embodiments, ACE is about 1.2 or more. In still other embodiments, ACE is about 2.0 or more. In other embodiments, ACE is about 3.0 or more. In yet other embodiments, ACE is about 4.0 or more.

The compositions of the present disclosure also have comparable to better time to peak arginine plasma concentration after administration with an effective dose. For example, the time to peak arginine plasma concentration may be about 120 minutes or less after dosing. In some aspects, the time to peak arginine plasma concentration may be about 60 minutes or less after dosing. Without being bound to any particular theory, a quicker time to arginine peak translates into quicker nitric oxide production.

As such, administration of the precursors discussed herein (or a composition include the precursor) provide a way to efficiently increase arginine concentrations in the body. Since, arginine leads to local production of NO, the increased arginine concentration will, in turn, increase NO production.

### EXAMPLES

The following non-limiting examples are merely illustrative of the preferred embodiments of the present invention, and are not to be construed as limiting the invention, the scope of which is defined by the appended claims.

### Example 1: Compositions

Several example compositions made in accordance with the present disclosure are provided below:

TABLE 1
Example Compositions

| | Dose (g / mg per capsule or scoop) | | | | | | | | |
|---|---|---|---|---|---|---|---|---|---|
| Ingredient | Ex A | Ex B | Ex C | Ex D | Ex E | Ex F | Ex G | Ex H | Ex I |
| Simulating | 2 g | — | 1 g | 2 g | 500 | 2 g | 750 | 750 | 500 |
| Agent 1 | | | | | mg | | mg | mg | mg |
| Stimulating | — | 2 g | 1 g | 500 g | 2 g | 1 g | — | — | 500 |
| Agent 2 | | | | | | | | | mg |
| Additive 1 | — | — | — | — | — | — | 750 | 750 | 750 |
| | | | | | | | mg | mg | mg |
| Additive 2 | | | | | | | | 2 g | 1 g |

The composition may include one or more stimulating agents disclosed herein or one or more additives. For example, the stimulating agent 1 and/or stimulating agent 2 in Table 1 above may be any of the citrulline and/or glutamine salts previously discussed. Additive 1 may be a creatine derivative as previously discussed. Additive 2 may be an antioxidant as previously discussed.

The compositions may be in capsule or powder form. For instance, Examples A and B may be administered once a day with a serving size of 2 capsules (1 g per capsule). Examples A and B may also be formulated in powder form and be administered once or twice a day (1 g per scoop). Examples C-I may be administered once a day by mixing one or two scoops with 8-12 ounces of water or other liquid suitable for ingestion. Examples C-I may also be formulated in capsule and taken once a day.

### Example 2: Solubility Determination

The saturated solubility of the certain stimulating agents discussed herein in deionized water was determined by adding increasing amounts at 62° F. More specifically, L-citrulline (control) was compared to citrulline HCl, a 1:1 blend of L-citrulline and creatine HCl, a 1:1 blend of citrulline HCl and creatine HCl using the procedure below. The solubility of creatine HCl was also obtained using the procedure below.
1. Add five grams of conjugate or blend into 100 mL of water in a 250 ml Beaker.
2. Record temperature of water before adding conjugate or blend.
3. After conjugate is added to water, stir or swirl solution for 2 minutes exactly.
4. If the solution is still cloudy or precipitate is present after 2 minutes, promptly transfer mixture into filter.
5. If the solution is not cloudy and all of conjugate appears to have gone into solution, add another five grams of conjugate and repeat on from step 3.
6. Record all data and amounts of conjugate added.
7. Once solution with precipitate has been filtered, dry the filter with conjugate and use mass by difference to record dry mass of conjugate.

### L-Citrulline

After five grams of L-citrulline was added to the water, the solution remained cloudy throughout the two-minute check point. The solution was filtered, dried, and massed. Only 0.02 g of the original five grams of L-citrulline was recovered un-dissolved by the water. The solubility was calculated to be 49.8 mg/ml at 62° F.

### Citrulline HCl

After the five grams of citrulline HCl was added to the water, the solution cleared after 70 seconds and remained clear throughout the two-minute checkpoint. Another five grams of citrulline HCl was added to the solution, which cleared after 90 seconds. After another five grams of citrulline HCl was added, the solution remained cloudy through the two-minute check point. The solution was filtered, dried, and massed. 0.09 grams were recovered in the dried filter. The solubility was calculated to be 149.1 mg/mL of citrulline HCl in water at 62° F. (about 3 times that of L-citrulline).

### Creatine HCl

After five grams of creatine HCl was added to the water, the solution cleared after 30 seconds and remained clear throughout the two-minute checkpoint. After 18 more additions of five grams of the blend was added (90 g), the solution cleared after 1 minute 50 seconds, but there were visible undissolved granules. The solution was filtered, dried, and massed. 0.09 grams were recovered in the dried filter. The solubility was calculated to be 949.1 mg/mL of the blend in water at 62° F.

### Citrulline HCl and Creatine HCl Blend

After five total grams of a blend of citrulline HCl (2.5 g) and creatine HCl (2.5 g) (“blend”) was added to the water, the solution cleared after 1 second and remained clear throughout the two-minute checkpoint. Another five grams of the blend was added to the solution, which cleared after 66 seconds. After ten more additions of five grams of the blend was added (50 g), the solution remained cloudy through the two-minute check point. The solution was filtered, dried, and massed. 0.12 grams were recovered in the dried filter. The solubility was calculated to be 598.8 mg/mL of the blend in water at 62° F. (about 12 times that of L-citrulline and about 4 times that of citrulline HCl).

### L-Citrulline and Creatine HCl Blend

After five total grams of a blend of L-citrulline (2.5 g) and creatine HCl (2.5 g) (“L-blend”) was added to the water, the solution cleared almost immediately and remained clear throughout the two-minute checkpoint. Another five grams of the L-blend was added to the solution, which cleared after 0.88 seconds. After two more additions of five grams of the L-blend was added (10 g), the solution remained cloudy through the two-minute check point. The solution was filtered, dried, and massed. 2.19 grams were recovered in the dried filter. The solubility was calculated to be 178.1 mg/mL of the blend in water at 62° F. (about 3.6 times that of L-citrulline and about 1.2 times that of citrulline HCl).

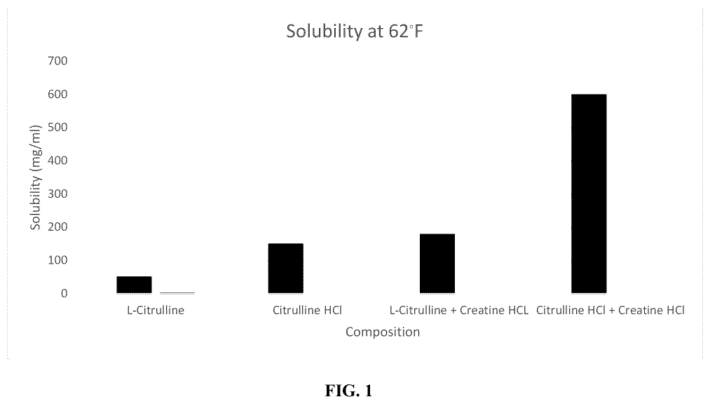

Since the human body is primarily an aqueous-based organism, the solubility results (as depicted in FIG. 1) predict a dramatic difference in the bioavailability between various conjugated dosage forms of an oral blend of Citrulline HCL and Creatine HCl.

### Example 3: Urine Arginine and Citrulline Levels Following Stimulating Agent Dosing

The subjects in each of Groups A and B were given a single 6 g dose (powder dissolved in about 4 oz of water). A urine sample is taken from each subject prior to dosing (baseline) and 24 hours after dosing. The pre-dosing sample for each subject is used as a baseline from which to compare any increase or decrease in arginine and/or citrulline urine levels.
Group A (8 subjects): 6 g of citrulline HCl in 4 oz. of water
Group B (8 subjects): 6 g of L-Citrulline in 4 oz. of water

A third group, consisting of seven subjects, were given a single 2 g dose, otherwise following the same protocol:
Group C (7 subjects): 2 g of citrulline HCl in 4 oz. of water

Internal standard (IS) solutions of arginine-13C6 and citrulline-d7 were prepared in 6 percent trichloroacetic acid solution. Standard substances of arginine and citrulline HCl were precisely weighed and dissolved in Sigmatrix Urine Diluent to make their stock solutions, at 5 mM for each compound. The two stock solutions were then mixed at an equal volume and the mixed standard solution was step by step diluted with Sigmatrix Urine Diluent to have nine serially diluted calibration solutions. The concentrations of each compound in the calibration solutions ranged from 0.015 to 250 μM.

Urine samples were thawed at room temperature. After vortex-mixing for 5 s, 100 μL of each sample was precisely aliquoted into a 1.5-mL Eppendorf safe-lock tube. A mixed urine sample was pooled from 100 μL of each of 10 randomly selected among all the urine samples. Nine 100-μL aliquots of this pooled urine sample were processed along with the batch samples and the calibration solutions for the purpose of quality control (QC) to monitor the analytical variations of the sample preparation and the assay.

To 100 μL of urine or 100 μL of each calibration solution, 100 μL of the IS solution was added. The mixtures were vortexed for 10 s at 3,000 rpm and then sonicated in an ice-water bath for 1 min, followed by centrifugal clarification at 21,000 g and 10° C. for 10 min. 100 μL of the clear supernatant of each solution was mixed with 900 μL of water. 4-μL aliquots of the resultant sample and calibration solutions were injected to run liquid chromatography-multiple reaction monitoring mass spectrometry (LC-MRM/MS) on an Agilent 1290 UHPLC system connected to an Agilent 6495B triple-quadrupole mass spectrometer, which was operated with positive-ion electrospray ionization and in the MRM mode using two pairs of MRM ion transitions and other MS parameters for arginine and citrulline, respectively.

A Waters HSS T3 UPLC column (2.1*100 mm, 1.8 μm) was used for the chromatographic separation and a 5-mM heptafluorobutyric acid solution and acetonitrile were used as the mobile phase for binary-solvent gradient elution under optimized conditions. LC-MRM/MS raw data files were acquired and processed using the Agilent MassHunter® software suite. For concentration calculations, linearly regressed calibration curves of arginine and citrulline were constructed respectively with the data acquired from the calibration solutions. Concentrations of the two compounds detected in urine were calculated by interpolating the calibration curve with the peak area ratios areas measured from the sample solutions, in an appropriate concentration range for each compound.

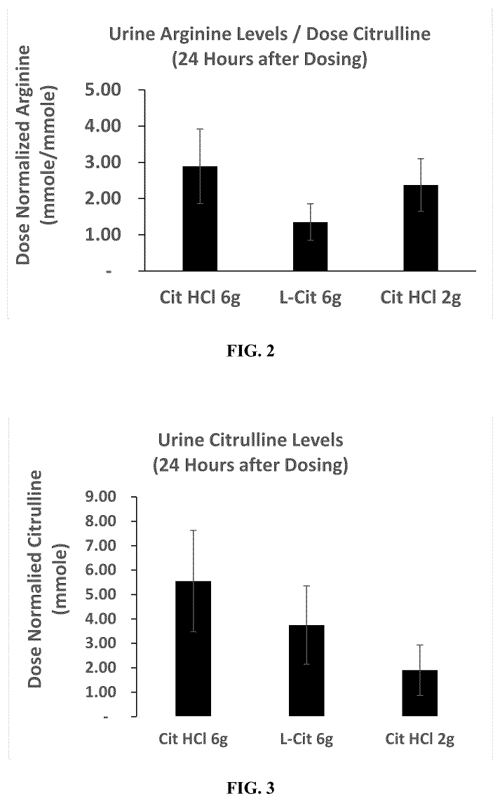

Table 2 and FIG. 2 demonstrates the average arginine in urine for each group.

TABLE 2
Arginine Urine Levels

| | Total Average Amount | |
|---|---|---|
| | (μM) | |
| Stimulating Agent | Baseline | 24 hours |
| Group A (6 g Citrulline HCl) | 14,789.25 | 80,919.23 |
| Group B (6 g L-Citrulline) | 8,397.56 | 46,233.58 |
| Group C (2 g Citrulline HCl) | 8,356.54 | 22,308.39 |

The percent increase in urine arginine 24 hours after dosing is higher per gram with Group C (about 133 percent/g) as compared to Groups A and B (about 91 percent/g).

Table 3 and FIG. 3 demonstrates the average citrulline in urine for each group.

TABLE 3
Citrulline Urine Levels

| | Total Average Amount | |
|---|---|---|
| | (μM) | |
| Stimulating Agent | Baseline | 24 hours |
| Group A (6 g Citrulline HCl) | 8,773.04 | 157,053.26 |
| Group B (6 g L-Citrulline) | 2,042.91 | 128,215.41 |
| Group C (2 g Citrulline HCl) | 2,471.80 | 17,880.65 |

The percent increase in urine citrulline 24 hours after dosing is much higher per gram with Group B (about 1000 percent/g) as compared to Groups A and C (about 300 percent/g). Taken together with the percent increase in urine arginine 24 hours after dosing, the data demonstrates that a lower dose of citrulline HCl (a direct precursor) provides efficient production of arginine in the body without also producing an excess of citrulline to be excreted.

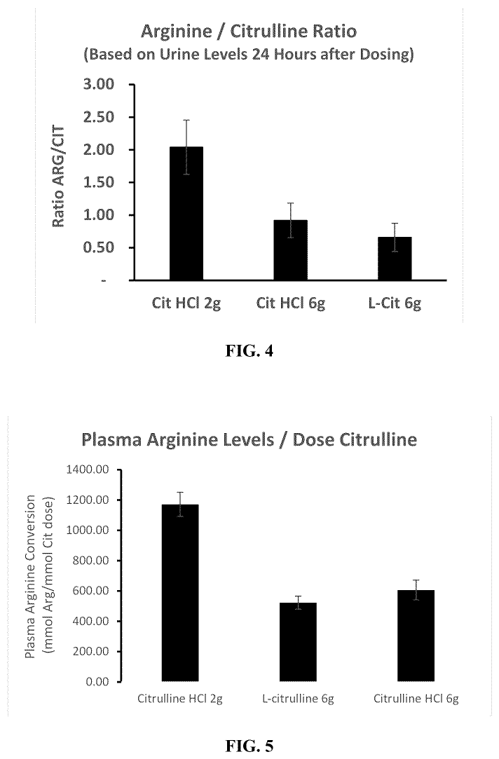

In this regard, the ACEᵤᵣᵢₙₑ value, i.e.,

$${ACE}_{urine} = \frac{{Arg}_{ur}}{{Cit}_{ur}} \qquad ( I )$$

where Argᵤᵣ and Citᵤᵣ represent the mean arginine and citrulline levels in the urine 24 hours after dosing (dose normalized), is shown below in Table 4 and graphically illustrated in FIG. 4.

TABLE 4
Arginine/Citrulline (Urine)

| | ACEᵤᵣᵢₙₑ | |
|---|---|---|
| Group | (mean) | Std Dev |
| A (6 g Citrulline HCl) | 0.92 | 0.26 |
| B (6 g L-Citrulline) | 0.66 | 0.22 |
| C (2 g Citrulline HCl) | 2.04 | 0.41 |

### Example 4: Plasma Arginine and Citrulline Levels Following Stimulating Agent Dosing

Plasma samples from each of Groups A, B, and C were taken from each subject prior to dosing (baseline) and at 30 minute intervals thereafter up to 180 minutes after dosing (30, 60, 90, 120, and 180 minutes). The pre-dosing sample for each subject is used as a baseline from which to compare any increase or decrease in arginine and/or citrulline plasma levels.

Internal standard (IS) solution of arginine-13C6 and citrulline-d7 were prepared in 6 percent trichloroacetic acid solution. Standard substances of arginine and citrulline were precisely weighed and dissolved in SigMatrix Ultra Serum Diluent to make their stock solutions, at 2000 or 4000 UM for each compound. The stock solutions were then mixed and further diluted with SigMatrix Ultra Serum Diluent to have 9 serially diluted calibration solutions. The concentrations of each compound in the calibration solutions ranged from 0.0008 to 50 μM.

Plasma samples were thawed at room temperature. After vortex-mixing for 5 s, 50 μL of each sample was precisely aliquoted into a 1.5-mL Eppendorf safe-lock tube. A mixed plasma sample was pooled from 50 μL of each of 20 randomly selected among all the 210 plasma samples. Twelve 50-μL aliquots of this pooled plasma sample were processed in parallel and analyzed periodically, along with the preparation and analysis of the batch samples and the calibration solutions for the purpose of quality control (QC) to monitor the analytical variations of the sample preparation and the assay.

To 50 μL of plasma or 50 μL of each calibration solution, 100 μL of the IS solution was added. The mixtures were vortexed for 10 s at 3,000 rpm and then sonicated in an ice-water bath for 1 min, followed by centrifugal clarification at 21,000 g and 10° C. for 10 min. 50 μL of the clear supernatant of each solution or each calibration solution was mixed with 200 μL of water. 4-μL aliquots of all the resultant solutions were injected to run liquid chromatography-multiple reaction monitoring mass spectrometry (LC-MRM/MS) on an Agilent 1290 UHPLC system connected to an Agilent 6495B triple-quadrupole mass spectrometer, which was operated with positive-ion electrospray ionization and in the MRM mode using two pairs of MRM ion transitions for each compound.

A Waters HSS T3 UPLC column (2.1*100 mm, 1.8 μm) was used for the chromatographic separation and a 5-mM heptafluorobutyric acid solution and acetonitrile were used as the mobile phase for binary-solvent gradient elution under optimized conditions. LC-MRM/MS raw data files were acquired and processed using the Agilent MassHunter® software suite. For concentration calculations, linearly regressed calibration curves of arginine and citrulline were constructed respectively with the data acquired from the calibration solutions.

Table 5 provides the arginine plasma levels (AUC per citrulline dose) for each subject in each group, along with the mean and standard deviation.

TABLE 5
Arginine Plasma Levels

| | Subject | | | | | | | |
|---|---|---|---|---|---|---|---|---|
| Group | 1 | 2 | 3 | 4 | 5 | 6 | 7 | 8 |
| A (6 g Citrulline HCl) | 506.35 | 862.79 | 353.81 | 640.59 | 555.06 | 409.14 | 729.65 | 790.10 |
| | | | | | | | Mean | 605.94 |
| | | | | | | | Std Dev | 64.91 |
| B (6 g L-Citrulline) | 444.22 | 642.15 | 550.94 | 428.62 | 417.72 | 381.21 | 690.32 | 629.92 |
| | | | | | | | Mean | 523.13 |
| | | | | | | | Std Dev | 42.84 |
| C (2 g Citrulline HCl) | 1189.73 | 1482.91 | 1175.79 | 929.50 | 1165.09 | 1020.04 | 1236.29 | — |
| | | | | | | | Mean | 1171.34 |
| | | | | | | | Std Dev | 79.40 |

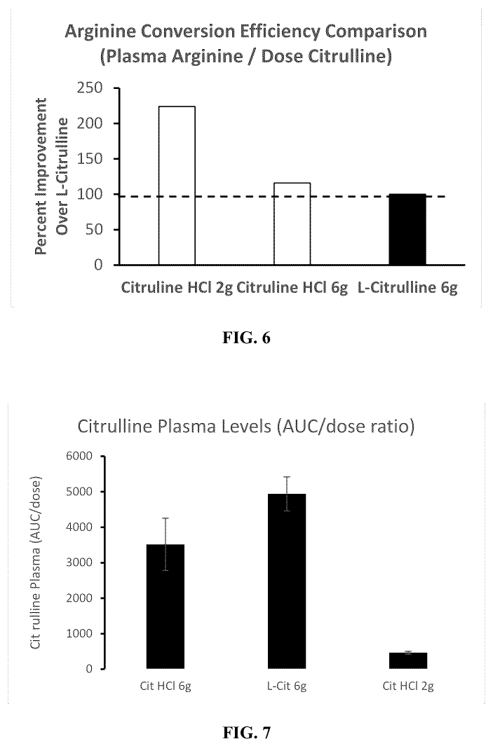

FIG. 5 compares the arginine plasma levels (normalized per dose) for Groups A-C. The group receiving 2 g of citrulline HCl had the highest arginine production per dose. FIG. 6 illustrates that the arginine plasma level for Group C was 223 percent higher than the arginine plasma level in Group B. The arginine plasma level for Group A was 115 percent of the arginine plasma level for Group B.

Table 6 provides the citrulline plasma levels (AUC per citrulline dose) for each subject in each group, along with the mean and standard deviation.

TABLE 6
Citrulline Plasma Levels

| | Subject | | | | | | | |
|---|---|---|---|---|---|---|---|---|
| Group | 1 | 2 | 3 | 4 | 5 | 6 | 7 | 8 |
| A (6 g Citrulline HCl) | 2264.16 | 4843.02 | 317.72 | 3799.57 | 4297.91 | 2536.80 | 2814.13 | 7281.31 |
| | | | | | | | Mean | 3519.32 |
| | | | | | | | Std Dev | 737.84 |
| B (6 g L-Citrulline) | 5386.04 | 5944.26 | 5759.24 | 4143.37 | 4736.92 | 2991.96 | 3535.38 | 7028.00 |
| | | | | | | | Mean | 4940.65 |
| | | | | | | | Std Dev | 480.72 |
| C (2 g Citrulline HCl) | 466.33 | 443.36 | 634.07 | 483.64 | 447.48 | 440.56 | 347.84 | — |
| | | | | | | | Mean | 466.18 |
| | | | | | | | Std Dev | 38.93 |

As can be seen in this data and corresponding FIG. 7, Group B has a much larger amount of citrulline in plasma than Group Groups A or C. The excess citrulline in the plasma not only indicates a lower conversion efficiency to arginine for Group B, it also results in excess citrulline that will need to be excreted.

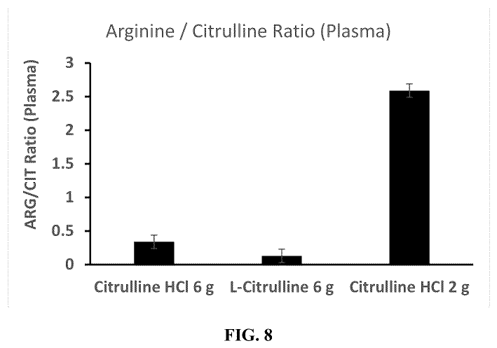

In this regard, the ACEₚₗₐₛₘₐ value, i.e.,

$${ACE}_{plasma} = \frac{{Arg}_{pl}}{{Cit}_{pl}} \qquad ( I ( b ) )$$

where Argₚₗ and Citₚₗ represent the mean arginine and citrulline levels in plasma (dose normalized), is shown below in Table 7 and graphically illustrated in FIG. 8.

TABLE 7
Arginine/Citrulline (Plasma)

| | ACEₚₗₐₛₘₐ | |
|---|---|---|
| Group | (mean) | Std Dev |
| A (6 g Citrulline HCl) | 0.34 | 0.13 |
| B (6 g L-Citrulline) | 0.13 | 0.02 |
| C (2 g Citrulline HCl) | 2.59 | 0.30 |

Although the present invention has been described with reference to a method of delivering arginine to the systemic circulation and, in turn, increasing NO levels in the body, it will be understood to those skilled in the art that the invention is capable of a variety of alternative embodiments within the spirit of the appended claims. For example, the compositions of the present disclosure are also contemplated for use to stimulate muscle protein synthesis and/or maintain protein homeostasis. In addition, the compositions of the present disclosure may be administered to decrease the risk of postoperative pulmonary hypertension. Moreover, administration of the compositions of the present disclosure to patients affected by sickle cell disease may produce a systemic vasodilator antihypertensive effect. Finally, the compositions of the present disclosure may be orally administered to improve cardiac performance with exercise.

What is claimed is:

1. A composition for increasing arginine physiological levels in a subject, which comprises a therapeutically effective amount of a stimulating agent, wherein the stimulating agent comprises a pharmaceutically acceptable salt of a direct arginine precursor, an indirect arginine precursor, or a combination thereof, and wherein the stimulating agent has an aqueous solubility about 120 mg/ml to about 200 mg/ml in water at 62° F.

2. The composition of claim 1, wherein the direct arginine precursor comprises citrulline.

3. The composition of claim 2, wherein the stimulating agent comprises citrulline hydrochloride.

4. The composition of claim 1, further comprising an antioxidant.

5. The composition of claim 4, wherein the antioxidant comprises betalain.

6. The composition of claim 1, further comprising a creatine derivative.

7. The composition of claim 6, wherein the creatine derivative comprises creatine hydrochloride.

8. The composition of claim 7, wherein the composition has an aqueous solubility about 500 mg/ml to about 1000 mg/ml in water at 62° F.

9. The composition of claim 1, comprised in an oral dosage form.

10. The composition of claim 1, wherein the oral dosage form is a powder.

11. The composition of claim 1, wherein the oral dosage form is a pill, capsule, tablet, or liquid.

12. A method of increasing arginine physiological levels in an animal comprising:
providing a composition comprising a stimulating agent, wherein the stimulating agent comprises a pharmaceutically acceptable salt of a direct arginine precursor, an indirect arginine precursor, or a combination thereof, and wherein the stimulating agent has an aqueous solubility of at least two times that of L-citrulline; and
administering an effective dose of the composition to the animal, wherein the composition has an arginine conversion efficiency according to (I(b)):
$${ACE}_{plasma} = \frac{{Arg}_{pl}}{{Cit}_{pl}} \geq 0.3 \qquad ( I ( b ) )$$
wherein Argₚₗ represents the dose normalized level of arginine in plasma and Citₚₗ represents the dose normalized level of citrulline in plasma.

13. The method of claim 12, wherein the direct arginine precursor comprises citrulline.

14. The method of claim 13, wherein the stimulating agent comprises citrulline hydrochloride.

15. The method of claim 12, wherein the composition is comprised in an oral dosage form.

16. The method of claim 15, wherein the oral dosage form is a powder.

17. The method of claim 16, wherein the oral dosage form is a pill, capsule, tablet, or liquid.

18. The method of claim 12, wherein the composition further comprises at least one additive comprising one or more antioxidants, creatine derivatives, or combinations thereof.

19. The method of claim 18, wherein the additive comprises creatine hydrochloride and the composition has an aqueous solubility of about 500 mg/ml to about 1000 mg/ml in water at 62° F.

20. The method of claim 12, wherein
$${ACE}_{plasma} = \frac{{Arg}_{pl}}{{Cit}_{pl}} \geq 1. .$$

21. A method of increasing nitric oxide physiological levels in an animal comprising:
providing a composition comprising an effective dose of stimulating agent, wherein the stimulating agent comprises a pharmaceutically acceptable salt of a direct arginine precursor and has an aqueous solubility of about 120 mg/ml to about 200 mg/ml in water at 62° F.; and
administering the composition to the animal, wherein the composition has an arginine conversion efficiency according to (I(b)):
$${ACE}_{plasma} = \frac{{Arg}_{pl}}{{Cit}_{pl}} \geq 0.15 \qquad ( I ( b ) )$$
wherein Argₚₗ represents the dose normalized level of arginine in plasma and Citₚₗ represents the dose normalized level of citrulline in plasma.

22. The method of claim 21, wherein the composition is comprised in an oral dosage form.

23. The method of claim 21, wherein the effective dose ranges from about 1000 mg to about 3000 mg.

24. The method of claim 21, wherein the stimulating agent comprises citrulline hydrochloride.

25. The method of claim 24, wherein the composition further comprises creatine hydrochloride, and wherein the composition has an aqueous solubility of about 500 mg/ml to about 1000 mg/ml in water at 62° F.
